(19) United States
(12) Patent Application Publication
(10) Pub. No.: US 2009/0023682 A1
(43) Pub. Date: Jan. 22, 2009

(54) Composition Comprising Dietary Fat Complexer and Methods of Using Same

(76) Inventors: Joseph Artiss, Windsor (CA); Catherine Jen, Bloomfield, MI (US)

Correspondence Address: CAHN & SAMUELS LLP, 1100 17th STREET NW, SUITE 401, WASHINGTON, DC, 20036, US

(21) Appl. No.: 12/127,263

(22) Filed: May 27, 2008

Related U.S. Application Data
(60) Provisional application No. 60/950,669, filed on Jul. 19, 2007.

(51) Int. Cl. A61K 31/724; A61P 3/04

(52) U.S. Cl. 514/58

(57) ABSTRACT

A composition includes α-cyclodextrin and at least one lipase inhibitor. A method for promoting weight loss, inhibiting weight gain, or maintaining weight in a subject in need thereof includes administering α-cyclodextrin and at least one lipase inhibitor to the subject, thereby reducing the bioavailability of ingested fat and reducing the unpleasant gastrointestinal side effects.

This application claims priority of U.S. Ser. No. 60/950,669, filed on Jul. 19, 2007, the entirety of which is incorporated herein by reference.

### I. FIELD OF THE INVENTION

The present invention relates to compositions comprising α-cyclodextrin and at least one lipase inhibitor and to methods of their use.

### II. BACKGROUND OF THE INVENTION

At about 9 kcal/g, fat is the most calorie dense food that people consume. Fat is used herein to refer to mostly triglycerides.

For fat or triglycerides to be absorbed, they must first be hydrolyzed to free fatty acids and monoglycerides. Triglycerides are not absorbed until they are first reduced to these free fatty acids and monoglycerides by enzymatic action of a lipase. In addition, fat droplets from the stomach must be dispersed into very small micelle structures by bile salts, released from the gall bladder, in order to increase the surface area of the hydrophobic fat so that the hydrophilic lipase enzyme can act upon it. Any composition that inhibits at least one of the lipase activity or the formation of the micelles will also inhibit triglyceride absorption.

The weight loss drug Orlistat for example (also known as tetrahydrolipstatin and sold under the brand name XENICAL® or ALLI is an inhibitor of gastrointestinal lipases, i.e. lipases (gastric lipase, carboxylester lipase, pancreatic lipase) that are responsible for breaking down ingested fat. Orlistat inhibits about ⅓ of normal lipase activity.

In addition, Orlistat requires a user to go on a very low fat diet and hence a low calorie diet in order to take the drug. Thus, if the user does not severely restrict fat intake, free fat will pass into the large intestine where it will cause unpleasant gastrointestinal side effects such as severe cramps, leaky diarrhea, oil discharge, fatty/oily stools, fecal urgency, increased defecation, and fecal incontinence. Other lipase inhibitors in pre-clinical or early clinical stages suffer the same drawbacks as Orlistat. However, the obese people that weight loss drugs such as Orlistat are designed to help are often the least likely to maintain a low fat and/or low calorie diet.

A product comprising α-cyclodextrin is commercially available as FBCx® from ArtJen Complexus Holdings Corp. of Windsor, Ontario, CANADA (http://www.fbcx.com). Taken as directed, FBCx® binds to and eliminates approximately 50-60% of the fat from a typical American diet. Unlike Orlistat, which allows free fat to pass into the large intestine and be metabolized, α-cyclodextrin forms a very stable complex with fat, thereby preventing lipolytic activity in the small intestine and microbial action in the large intestine. The FBCx®-fat complex is eventually passed unnoticed in the feces. Accordingly, the unwanted gastrointestinal side effects that occur with the use of lipase inhibitors are not present. Further, a user is not required to go on a very low fat diet in order to take FBCx®.

U.S. Patent Application Publication 2006/0269510 A1 discloses a pharmaceutical combination or composition comprising a lipase inhibitor, preferably Orlistat, and a bile acid sequestrant for treating obesity. The bile acid sequestrant may be β-cyclodextrin or γ-cyclodextrin. See also U.S. Patent Application Publication 2005/0136030 A1; U.S. Patent Application Publication 2004/0105838 A1; U.S. Pat. No. 6,562,329 and U.S. Pat. No. 6,756,364.

U.S. Pat. No. 6,890,549 B2 to Artiss et al. relates to fat containing consumable food products comprising α-cyclodextrin. The food products have reduced levels of bioavailable fat but have substantially the same fat, cholesterol and caloric content as a like food without α-cyclodextrin. The invention also relates to methods for reducing the bioavailability of fats in fat containing food products without reducing caloric content and to methods for reducing low-density lipoproteins in a subject and reducing or controlling weight by administering the food products of this invention. U.S. Pat. No. 6,890,549 B2 is incorporated herein by reference in its entirety.

### III. SUMMARY OF THE INVENTION

It is an object of the present invention to provide a composition that is easy to administer to a subject in need of weight loss or inhibition of weight gain.

An advantage of the present invention is that the unpleasant gastrointestinal side effects of lipase inhibitors are greatly reduced or eliminated.

Another advantage of the present invention is that a user of the composition is not required to go on a low fat diet.

The above objects and advantages are satisfied by a composition comprising α-cyclodextrin and at least one lipase inhibitor.

As used herein “substantially”, “generally”, “relatively”, “approximately”, and “about” are relative modifiers intended to indicate permissible variation from the characteristic so modified. It is not intended to be limited to the absolute value or characteristic which it modifies but rather approaching or approximating such a physical or functional characteristic.

In this detailed description, references to “one embodiment”, “an embodiment”, or “in embodiments” mean that the feature being referred to is included in at least one embodiment of the invention. Moreover, separate references to “one embodiment”, “an embodiment”, or “in embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

### IV. DETAILED DESCRIPTION OF THE INVENTION

The present invention relates to compositions comprising α-cyclodextrin and at least one lipase inhibitor and to methods of their use. According to the present invention, a subject or individual in need of weight loss or inhibition of weight gain is administered α-cyclodextrin and at least one lipase inhibitor for a time sufficient to produce weight loss or inhibition of weight gain. The α-cyclodextrin and the at least one lipase inhibitor reduce the bioavailability of ingested fat.

In embodiments, the α-cyclodextrin and the lipase inhibitor may be administered to the subject as separate compositions, preferably at substantially the same time. Alternatively, the α-cyclodextrin and the lipase inhibitor may be administered as a combined single composition.

In embodiments, the α-cyclodextrin may comprise FBCx® from ArtJen Complexus Holdings Corp. of Windsor, Ontario, CANADA. The α-cyclodextrin may be substantially pure α-cyclodextrin. In embodiments, the lipase inhibitor may comprise at least one of Orlistat, XENICAL®, or ALLI or other lipase inhibitors to be identified in the future. At least one of the α-cyclodextrin or the lipase inhibitor may be in a form selected from the group consisting of a pill, tablet, powder, capsule, liquid, cracker, wafer, or confection.

Without wishing to be bound by theory, the α-cyclodextrin acts first to form a very stable fat complex in the stomach and then pass into small intestine which prevents lipase from acting on the fat. The lipase inhibitor prevents at least a portion of the remaining unbound fat from being hydrolyzed by lipase. Accordingly, it is believed that the combination of α-cyclodextrin and lipase inhibitor advantageously eliminates a larger portion of fat than known weight loss compositions while also reducing or eliminating the unpleasant gastrointestinal side effects of lipase inhibitors.

The following non-limiting example is illustrative of the present invention.

### EXAMPLE

A healthy female in her fifties took 1 XENICAL® capsule (120 mg) and then ate 2 pieces of pizza and a bowl of ice cream. Within 1 hour, she had severe cramping and experienced gassy, explosive diarrhea for approximately one hour. When the diarrhea stopped, she continued to pass bright orange oil for about 2 hours. The next day, she repeated the meal with XENICAL but also took 2 FBCx® tablets (1 g each, total 2 g). She experienced no adverse side effects.

Although specific embodiments of the invention have been described herein, it is understood by those skilled in the art that many other modifications and embodiments of the invention will come to mind to which the invention pertains, having benefit of the teaching presented in the foregoing description and associated drawings.

It is therefore understood that the invention is not limited to the specific embodiments disclosed herein, and that many modifications and other embodiments of the invention are intended to be included within the scope of the invention. Moreover, although specific terms are employed herein, they are used only in generic and descriptive sense, and not for the purposes of limiting the description invention.

1. A composition, comprising:
α-cyclodextrin; and
at least one lipase inhibitor.

2. A composition according to claim 1, wherein the lipase inhibitor comprises tetrahydrolipstatin.

3. A composition according to claim 1, wherein the lipase inhibitor comprises at least one of Orlistat, XENICAL®, or ALLI.

4. A composition according to claim 1, wherein the α-cyclodextrin comprises FBCx®.

5. A composition according to claim 1, wherein the α-cyclodextrin is substantially pure α-cyclodextrin.

6. A composition according to claim 1, wherein the α-cyclodextrin and the lipase inhibitor are separate compositions.

7. A composition according to claim 1, wherein the α-cyclodextrin and the lipase inhibitor are a single composition.

8. A composition according to claim 1, wherein at least one of the α-cyclodextrin or the lipase inhibitor is in a form selected from the group consisting of a pill, tablet, powder, capsule, liquid, cracker, wafer, or confection.

9. A weight loss composition comprising α-cyclodextrin and Orlistat or ALLI.

10. A method for at least one of promoting weight loss, inhibiting weight gain, or maintaining weight in a subject in need thereof, comprising:
administering α-cyclodextrin and at least one lipase inhibitor to the subject, thereby reducing the bioavailability of ingested fat.

11. A method according to claim 10, wherein the α-cyclodextrin and the lipase inhibitor are administered to the subject separately.

12. A method according to claim 11, wherein the α-cyclodextrin and the lipase inhibitor are administered to the subject at substantially the same time.

13. A method according to claim 10, wherein the α-cyclodextrin and the lipase inhibitor are administered to the subject simultaneously.

14. A method according to claim 10, wherein the α-cyclodextrin and the lipase inhibitor are administered to the subject as a single composition.

15. A method according to claim 10, wherein at least one of cramps, diarrhea, or oil discharge are inhibited.
